(19) United States
(12) Patent Application Publication
(10) Pub. No.: US 2026/0200899 A1
(43) Pub. Date: Jul. 16, 2026

(54) SYNTHESIS OF CRAC CHANNEL INHIBITORS

(71) Applicant: CalciMedica, Inc., La Jolla, CA (US)

(72) Inventors: Kenneth STAUDERMAN, San Diego, CA (US); Michael DUNN, La Jolla, CA (US); Kay OLMSTEAD, San Diego, CA (US)

(21) Appl. No.: 19/435,448

(22) Filed: Dec. 29, 2025

Related U.S. Application Data
(63) Continuation of application No. 18/252,695, filed on Nov. 13, 2020, now Pat. No. 12,522,589.

(51) Int. Cl. C07D 405/04

(52) U.S. Cl. CPC C07D 405/04

(57) ABSTRACT

Provided herein is an improved synthetic method for the production of CRAC channel inhibitors. The synthetic method provides a method for producing highly pure CRAC channel inhibitors for clinical testing.

### CROSS REFERENCE TO RELATED APPLICATIONS

This application is a continuation of U.S. application Ser. No. 18/252,695, filed May 11, 2023, which is a U.S. National Stage Application filed under 35 U.S.C. § 371 of International Application No. PCT/IB2020/000965, filed on Nov. 13, 2020, each of which is hereby incorporated by reference in its entirety.

### BACKGROUND

Calcium plays a vital role in cell function and survival. For example, calcium is a key element in the transduction of signals into and within cells. Cellular responses to growth factors, neurotransmitters, hormones and a variety of other signal molecules are initiated through calcium-dependent processes.

Virtually all cell types depend in some manner upon the generation of cytoplasmic Ca²⁺ signals to regulate cell function, or to trigger specific responses. Cytosolic Ca²⁺ signals control a wide array of cellular functions ranging from short-term responses such as contraction and secretion to longer-term regulation of cell growth and proliferation. Usually, these signals involve some combination of release of Ca²⁺ from intracellular stores, such as the endoplasmic reticulum (ER), and influx of Ca²⁺ across the plasma membrane. In one example, cell activation begins with an agonist binding to a surface membrane receptor, which is coupled to phospholipase C (PLC) through a G-protein mechanism. PLC activation leads to the production of inositol 1,4,5-triphosphate (IP3), which in turn activates the IP3 receptor causing release of Ca²⁺ from the ER. The fall in ER Ca²⁺ then signals to activate plasma membrane store-operated calcium (SOC) channels.

Store-operated calcium (SOC) influx is a process in cellular physiology that controls such diverse functions such as, but not limited to, refilling of intracellular Ca²⁺ stores (Putney et al. Cell, 75, 199-201, 1993), activation of enzymatic activity (Fagan et al., J. Biol. Chem. 275:26530-26537, 2000), gene transcription (Lewis, Annu. Rev. Immunol. 19:497-521, 2001), cell proliferation (Nunez et al., J. Physiol. 571.1, 57-73, 2006), and release of cytokines (Winslow et al., Curr. Opin. Immunol. 15:299-307, 2003). In some nonexcitable cells, e.g., blood cells, immune cells, hematopoietic cells, T lymphocytes and mast cells, SOC influx occurs through calcium release-activated calcium (CRAC) channels, a type of SOC channel.

The calcium influx mechanism has been referred to as store-operated calcium entry (SOCE). Stromal interaction molecule (STIM) proteins are an essential component of SOC channel function, serving as the sensors for detecting the depletion of calcium from intracellular stores and for activating SOC channels.

### SUMMARY

Provided herein, in one aspect, is a process of synthesizing CRAC channel inhibitors of Formula (I):

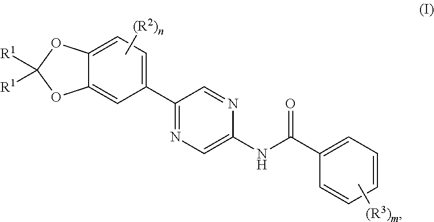

or a pharmaceutically acceptable salt thereof, wherein:
R¹, R², and R³ are independently selected at each occurrence from hydrogen, halogen, and C₁-C₃ alkyl optionally substituted with one or more substituents independently selected from hydrogen, halogen, —OH, —OR⁴, —CN, —N(R⁴)₂, and —NO₂;
or two R¹ groups are taken together with the atoms to which they are attached to form a carbocycle;
n is 0, 1, 2, or 3;
m is 0, 1, 2, 3, 4, or 5; and
R⁴ is independently selected at each occurrence from hydrogen; and C₁₋₆ alkyl, C₂₋₆ alkenyl, and C₂₋₆ alkynyl, each of which may be optionally substituted at each occurrence by halogen, —CN, —NO₂, —OH, —NH₂, and OCH₃;
   wherein the process comprises reacting a compound of Formula (I-A)

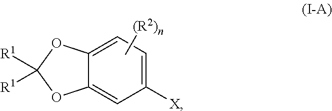

with a compound of Formula (I-B)

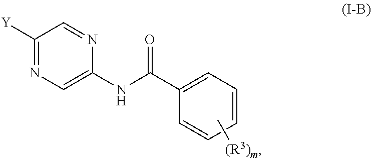

in the presence of a base, a catalyst, and a solvent, wherein:
X is selected from —B(OH)₂, —BF₃K,

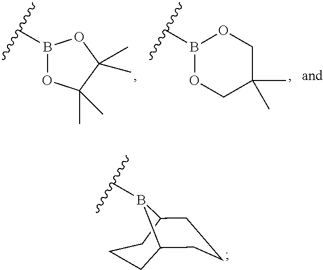

and
Y is selected from Cl, Br, and I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium phosphate.

In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄ (BF₄)₂, PdCl₂, XPhos-Pd-G₃, Pd-PEPPSI™—IPr, Pd-PEPPSI™—SIPr, and Pd-PEPPSI™-IPent. In some embodiments, the catalyst is Pd(PPh₃)₄.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is 1,4-dioxane.

In some embodiments, the compound of Formula (I-A), the compound of Formula (I-B), the base, the catalyst, and the solvent are stirred:
for no longer than 16 hours; and
at a temperature of between about 75° C. and about 80° C.

In some embodiments, the process further comprises precipitating the compound of Formula (I) and isolating it by filtration.

In some embodiments, the process provides the compound of Formula (I) in a synthetic yield of greater than about 75%.

In some embodiments, the compound of Formula (I-A)

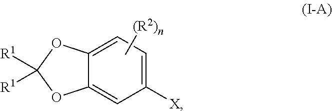

is synthesized by reacting a compound of Formula (I-C)

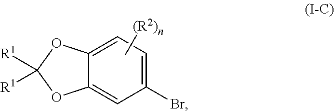

with a borylating agent in the presence of a base and a solvent.

In some embodiments, the borylating agent is selected from 2-isopropoxy-4,4,5,5, -tetramethyl-1,3,2-dioxaborolane, 2-methoxy-4,4,5,5, -tetramethyl-1,3,2-dioxaborolane, and 2-ethoxy-4,4,5,5, -tetramethyl-1,3,2-dioxaborolane. In some embodiments, the borylating agent is 2-isopropoxy-4,4,5,5, -tetramethyl-1,3,2-dioxaborolane.

In some embodiments, the base is selected from isopropylmagnesium chloride, isopropylmagnesium lithium chloride, methylmagnesium bromide, methylmagnesium chloride, methylmagnesium iodide, ethylmagnesium chloride, ethylmagnesium bromide, isopropylmagnesium bromide, methyllithium, ethyllithium, isopropyllithium, n-butyllithium, and tert-butyllithium. In some embodiments, the base is isopropylmagnesium lithium chloride.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is tetrahydrofuran.

In some embodiments, the compound of Formula (I-C), the borylating agent, the base, and the solvent are stirred:
for no longer than 2 hours; and
at a temperature of between about 0° C. and about 25° C.

In some embodiments, the process further comprises precipitating the compound of Formula (I-A) and isolating it by filtration.

In some embodiments, the process provides the compound of Formula (I-A) in a synthetic yield of greater than about 70%.

In some embodiments, the compound of Formula (I-C)

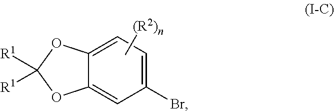

is synthesized by reacting a compound of Formula (I-D)

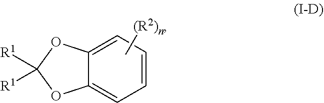

with a brominating agent in the presence of an acid and a solvent.

In some embodiments, the brominating agent is selected from N-bromosuccinimide, tribromoisocyanuric acid, 1,3-dibromo-5,5-dimethylhydantoin, and bromine. In some embodiments, the brominating agent is 1,3-dibromo-5,5-dimethylhydantoin.

In some embodiments, the acid is selected from hydrochloric acid, sulfuric acid, nitric acid, acetic acid, hydrobromic acid, phosphoric acid, formic acid, and trifluoroacetic acid. In some embodiments, the acid is sulfuric acid.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is acetonitrile.

In some embodiments, the compound of Formula (I-D), the brominating agent, the acid, and the solvent are stirred:
for no longer than 12 hours; and
at a temperature of between about 0° C. and about 15° C.

In some embodiments, the process further comprises extracting the compound of Formula (I-C) and isolating it by concentration. In some embodiments, the process provides the compound of Formula (I-C) in a synthetic yield of greater than about 80%.

In some embodiments, the compound of Formula (I-B)

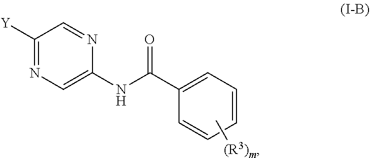

is synthesized by reacting a compound of Formula (I-E)

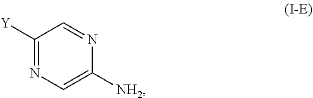

with a compound of Formula (I-F)

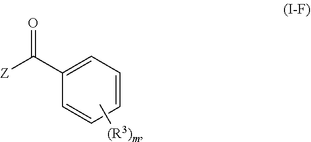

in the presence of a base, wherein:
Z is selected from Cl, Br, and I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is pyridine.

In some embodiments, the compound of Formula (I-E), the compound of Formula (I-F), and the base are stirred:
for no longer than 2 hours; and
at a temperature of between about 20° C. and about 25° C.

In some embodiments, the process further comprises precipitating the compound of Formula (I-B) and isolating it by filtration.

In some embodiments, the process provides the compound of Formula (I-B) in a synthetic yield of greater than about 80%.

In some embodiments, the compound of Formula (I-F)

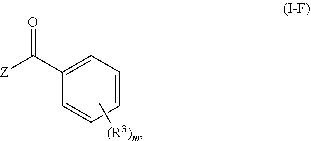

is synthesized by reacting a compound of Formula (I-G)

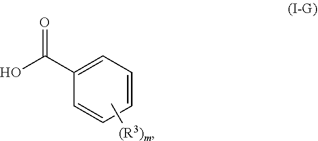

with an acyl halide preparation agent in the presence of a solvent.

In some embodiments, the acyl halide preparation agent is selected oxalyl chloride, thionyl chloride, phosphoryl chloride, phosphorous trichloride, phosphorous pentachloride, phosgene, diphosgene, triphosgene, and cyanuric chloride. In some embodiments, the acyl halide preparation agent is oxalyl chloride.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is tetrahydrofuran.

In some embodiments, the compound of Formula (I-G), the acyl halide preparation agent, and the solvent are stirred:
for no longer than 16 hours; and
at a temperature of between about 20° C. and about 25° C.

Provided herein, in another aspect, is a process of synthesizing CRAC channel inhibitors of Formula (II):

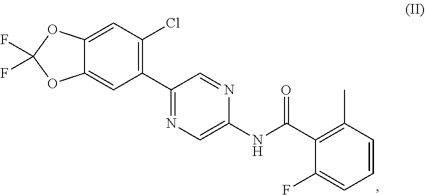 [SMILES: Cc1cccc(F)c1C(=O)Nc1cnc(-c2cc3c(cc2Cl)OC(F)(F)O3)cn1]

or a pharmaceutically acceptable salt thereof, wherein the process comprises reacting a compound of Formula (II-A)

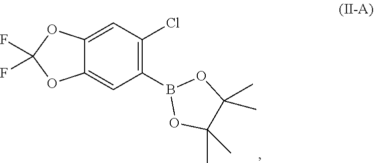 [SMILES: CC1(C)OB(c2cc3c(cc2Cl)OC(F)(F)O3)OC1(C)C]

with a compound of Formula (I-B)

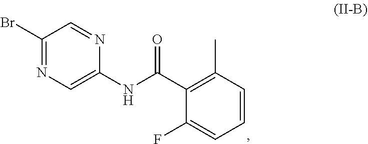 [SMILES: Cc1cccc(F)c1C(=O)Nc1cnc(Br)cn1]

in the presence of a base, a catalyst, and a solvent.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium phosphate.

In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄ (BF₄)₂, PdCl₂, XPhos-Pd-G₃, Pd-PEPPSI™—IPr, Pd—PEPPSI™—SIPr, and Pd-PEPPSI™-IPent. In some embodiments, the catalyst is Pd(PPh₃)₄.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is 1,4-dioxane.

In some embodiments, the compound of Formula (II-A), the compound of Formula (II-B), the base, the catalyst, and the solvent are stirred:
for no longer than 16 hours; and
at a temperature of between about 75° C. and about 80° C.

In some embodiments, the process further comprises precipitating the compound of Formula (II) and isolating it by filtration.

In some embodiments, the process provides the compound of Formula (II) in a synthetic yield of greater than about 75%.

In some embodiments, the compound of Formula (II-A)

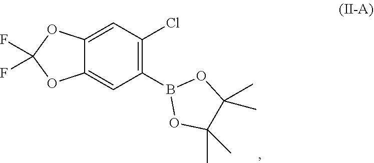 [SMILES: CC1(C)OB(c2cc3c(cc2Cl)OC(F)(F)O3)OC1(C)C]

is synthesized by reacting a compound of Formula (II-C)

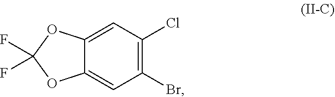 [SMILES: FC1(F)Oc2cc(Cl)c(Br)cc2O1]

with a borylating agent in the presence of a base and a solvent.

In some embodiments, the borylating agent is selected from 2-isopropoxy-4,4,5,5, -tetramethyl-1,3,2-dioxaborolane, 2-methoxy-4,4,5,5, -tetramethyl-1,3,2-dioxaborolane, and 2-ethoxy-4,4,5,5, -tetramethyl-1,3,2-dioxaborolane. In some embodiments, the borylating agent is 2-isopropoxy-4,4,5,5, -tetramethyl-1,3,2-dioxaborolane.

In some embodiments, the base is selected from isopropylmagnesium chloride, isopropylmagnesium lithium chloride, methylmagnesium bromide, methylmagnesium chloride, methylmagnesium iodide, ethylmagnesium chloride, ethylmagnesium bromide, isopropylmagnesium bromide, methyllithium, ethyllithium, isopropyllithium, n-butyllithium, and tert-butyllithium. In some embodiments, the base is isopropylmagnesium lithium chloride.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is tetrahydrofuran.

In some embodiments, the compound of Formula (II-C), the borylating agent, the base, and the solvent are stirred:
for no longer than 2 hours; and
at a temperature of between about 0° C. and about 25° C.

In some embodiments, the process further comprises precipitating the compound of Formula (II-A) and isolating it by filtration.

In some embodiments, the process provides the compound of Formula (II-A) in a synthetic yield of greater than about 70%.

In some embodiments, the compound of Formula (II-C)

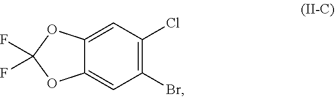 [SMILES: FC1(F)Oc2cc(Cl)c(Br)cc2O1]

is synthesized by reacting a compound of Formula (II-D)

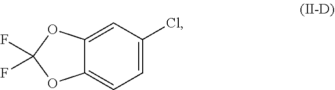 [SMILES: FC1(F)Oc2ccc(Cl)cc2O1]

with a brominating agent in the presence of an acid and a solvent.

In some embodiments, the brominating agent is selected from N-bromosuccinimide, tribromoisocyanuric acid, 1,3-dibromo-5,5-dimethylhydantoin, and bromine. In some embodiments, the brominating agent is 1,3-dibromo-5,5-dimethylhydantoin.

In some embodiments, the acid is selected from hydrochloric acid, sulfuric acid, nitric acid, acetic acid, hydrobromic acid, phosphoric acid, formic acid, and trifluoroacetic acid. In some embodiments, the acid is sulfuric acid.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is acetonitrile.

In some embodiments, the compound of Formula (II-D), the brominating agent, the acid, and the solvent are stirred:
for no longer than 12 hours; and
at a temperature of between about 0° C. and about 15° C.

In some embodiments, the process further comprises extracting the compound of Formula (II-C) and isolating it by concentration.

In some embodiments, the process provides the compound of Formula (II-C) in a synthetic yield of greater than about 80%.

In some embodiments, the compound of Formula (II-B)

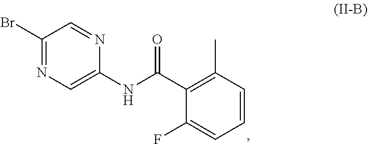 [SMILES: Cc1cccc(F)c1C(=O)Nc1cnc(Br)cn1]

is synthesized by reacting a compound of Formula (II-E)

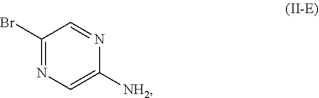

with a compound of Formula (II-F)

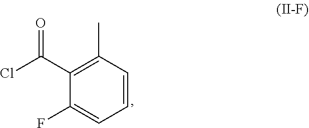

in the presence of a base, wherein:
Z is selected from Cl, Br, and I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is pyridine.

In some embodiments, the compound of Formula (II-E), the compound of Formula (II-F), and the base are stirred:
for no longer than 2 hours; and
at a temperature of between about 20° C. and about 25° C.

In some embodiments, the process further comprises precipitating the compound of Formula (II-B) and isolating it by filtration.

In some embodiments, the process provides the compound of Formula (II-B) in a synthetic yield of greater than about 80%.

In some embodiments, the compound of Formula (II-F)

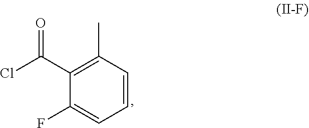

is synthesized by reacting a compound of Formula (II-G)

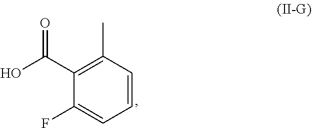

with an acyl halide preparation agent in the presence of a solvent.

In some embodiments, the acyl halide preparation agent is selected from oxalyl chloride, thionyl chloride, phosphoryl chloride, phosphorous trichloride, phosphorous pentachloride, phosgene, diphosgene, triphosgene, and cyanuric chloride. In some embodiments, the acyl halide preparation agent is oxalyl chloride.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is tetrahydrofuran.

In some embodiments, the compound of Formula (II-G), the acyl halide preparation agent, and the solvent are stirred:
for no longer than 16 hours; and
at a temperature of between about 20° C. and about 25° C.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

Unless defined otherwise, all technical and scientific terms used herein have the same meaning as is commonly understood in the field to which the claimed subject matter belongs. In the event that there is a plurality of definitions for terms herein, those in this section prevail. All patents, patent applications, publications, and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information is found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms are found in reference works, including but not limited to, Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed.

The term “CRAC channel inhibitor” refers to inhibitors that suppress calcium release activated channel (CRAC), which are specialized plasma membrane Ca²⁺ ion channels that slowly replenish depleted levels of calcium in the endoplasmic reticulum.

The terms “inhibits”, “inhibiting”, or “inhibitor” of CRAC channel activity, as used herein, refer to inhibition of store operated calcium channel activity or calcium release activated calcium channel activity.

As used herein, C₁-Cₓ includes C₁-C₂, C₁-C₃ . . . . C₁-Cₓ. C₁-Cₓ refers to the number of carbon atoms that make up the moiety to which it designates (excluding optional substituents).

An “alkyl” group refers to an aliphatic hydrocarbon group. The alkyl groups may or may not include units of unsaturation. The alkyl moiety may be a “saturated alkyl” group, which means that it does not contain any units of unsaturation (i.e. a carbon-carbon double bond or a carbon-carbon triple bond). The alkyl group may also be an “unsaturated alkyl” moiety, which means that it contains at least one unit of unsaturation. The alkyl moiety, whether saturated or unsaturated, may be branched, straight chain, or cyclic.

The “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, hexyl, propen-3-yl(allyl), cyclopropylmethyl, cyclobutylmethyl, cyclopentylmethyl, cyclohexylmethyl. Alkyl groups can be substituted or unsubstituted. Depending on the structure, an alkyl group can be a monoradical or a diradical (i.e., an alkylene group).

The term “alkenyl” refers to a type of alkyl group in which the first two atoms of the alkyl group form a double bond that is not part of an aromatic group. That is, an alkenyl group begins with the atoms —C(R)═CR₂, wherein R refers to the remaining portions of the alkenyl group, which may be the same or different. Non-limiting examples of an alkenyl group include CH═CH₂, —C(CH₃)═CH₂, —CH═CHCH₃, —CH═C(CH₃)₂ and —C(CH₃)═CHCH₃. The alkenyl moiety may be branched, straight chain, or cyclic (in which case, it would also be known as a “cycloalkenyl” group). Alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group. Non-limiting examples of an alkynyl group include —C≡CH, —C≡CCH₃, —C≡CCH₂CH₃ and —C≡CCH₂CH₂CH₃. The “R” portion of the alkynyl moiety may be branched, straight chain, or cyclic. An alkynyl group can have 2 to 6 carbons. Alkynyl groups can be substituted or unsubstituted. Depending on the structure, an alkynyl group can be a monoradical or a diradical (i.e., an alkynylene group).

“Carbocycle” refers to saturated, unsaturated or aromatic rings in which each atom of the ring is carbon. Carbocycle may be monocyclic or polycyclic and may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary carbocycles include cyclopropyl, cyclobutyl, cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

The term “halo” or, alternatively, “halogen” means fluoro, chloro, bromo, and iodo.

The compounds disclosed herein, in some embodiments, are used in different enriched isotopic forms, e.g., enriched in the content of ²H, ³H, ¹¹C, ¹³C and/or ¹⁴C. In one particular embodiment, a compound described herein is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of the present disclosure.

The compounds of the present disclosure optionally contain unnatural proportions of atomic isotopes at one or more atoms that constitute such compounds. For example, the compounds may be labeled with isotopes, such as deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds described herein, whether radioactive or not, are encompassed within the scope of the present disclosure.

In certain embodiments, the compounds disclosed herein have some or all of the 1H atoms replaced with 2H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)] 2000, 110 pp; George W.; Varma, Rajender S. The Synthesis of Radiolabeled Compounds via Organometallic Intermediates, Tetrahedron, 1989, 45 (21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

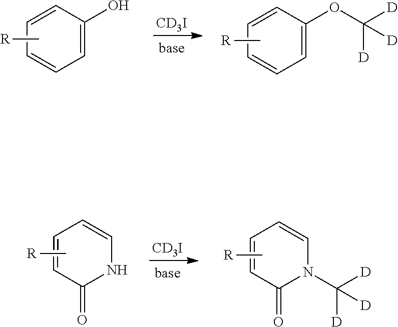

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

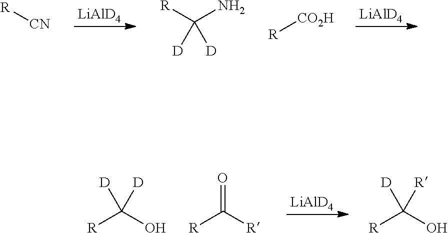

Deuterium gas and palladium catalysts are employed to reduce unsaturated carbon-carbon linkages and to perform a reductive substitution of aryl carbon-halogen bonds as illustrated, by way of example only, in the reaction schemes below.

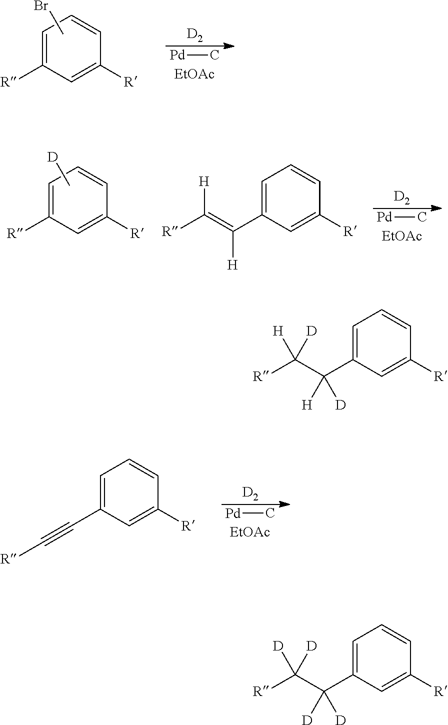

The term “nonpolar solvent” refers to a solvent that lacks a substantial electric dipole moment. Examples of “nonpolar solvent” as used herein include, but are not limited to, pentane, cyclopentane, hexane, cyclohexane, benzene, carbon tetrachloride, toluene, 1,4-dioxane, diethyl ether, and chloroform.

The term “aprotic polar solvent” refers to a solvent that lacks an acidic hydrogen atom. Intrinsically, an “aprotic polar solvent” does not facilitate hydrogen bonding interactions, aiding in S_{N}2-type reactions. Examples of “polar aprotic solvent” as used herein include, but are not limited to, chloroform, N-methylpyrrolidone, tetrahydrofuran, ethyl acetate, acetone, dimethylformamide, acetonitrile, dimethyl sulfoxide, propylene carbonate, and dichloromethane.

The term “protic polar solvent” refers to a solvent that has a labile, or acidic, hydrogen atom. “Protic polar solvent” facilitate hydrogen bonding interactions. Examples of “polar aprotic solvent” as used herein include, but are not limited to, water, acetic acid, formic acid, methanol, ethanol, n-propanol, and t-butanol.

The term “acid” refers to a molecule that has a labile, or acidic, hydrogen atom. Examples of “acid” as used herein include, but are not limited to, trifluoroacetic acid, 2, 2, 2-trifluoroethanol, sulfuric acid, nitric acid, hydrofluoric acid, hydrobromic acid, hydroiodic acid, triflic acid, perchloric acid, phosphoric acid, chloric acid, methanesulfonic acid, p-toluenesulfonic acid, acetic acid, formic acid, and hydrochloric acid.

The term “base” refers to a molecule that can extract a hydrogen atom from another molecule. Examples of “base” as used herein include, but are not limited to, lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine.

The term “coupling reaction” refers to a chemical reaction where two fragments combine with the aid of a metal catalyst, “catalyst”, or “coupling catalyst”. Examples of “coupling reactions” as used herein include, but are not limited to, Suzuki, Negishi, Stille, or Liebeskind-Srogl coupling reactions. Examples of “coupling catalysts” as used herein include, but are not limited to catalysts deriving from copper, palladium, nickel, or iron. The term “palladium-based catalyst” refers to a palladium-derived coupling catalyst used in coupling reactions. Examples of a “palladium-based catalyst” as used herein include, but are not limited to, Pd(PPh₃)₄, Pd(OAc)₂, Pd(dppf)Cl₂, Pd(dtpbf)Cl₂, Pd(dba)₂, Pd(PCy₃)₂, Pd(dppe)Cl₂, Pd(t-Bu₃P)₂, PdCl₂[P (o-Tol)₃]₂, benzylbis(triphenylphosphine)palladium(II) chloride, (A-Phos)₂Cl₂Pd, Na₂PdCl₄, and PdCl₂(PPh₃)₄.

The term “brominating agent” refers to a chemical reagent that is used to add bromine atoms to a, typically organic, reactant. Examples of “brominating agents” as used herein include, but are not limited to, bromine, bromine 1,4-dioxane complex, bromotrichloromethane, 1,2-dibromo-1,1,2,2, -tetrachloroethane, carbon tetrabromide, tetrabutylammonium tribromide, trimethylphenylammonium tribromide, benzyltrimethylammonium tribromide, pyridinium bromide perbromide, 4-dimethylaminopyridinium bromide perbromide, 1-butyl-3-methylimidazolium tribromide, 1,8-diazabicyclo[5.4.0]-7-undecene hydrogen tribromide, N-bromosuccinimide, N-bromophthalimide, N-bromosaccharin, N-bromoacetamide, 2-bromo-2cyano-N,N-dimethylacetamide, 1,3-dibromo-5,5-dimethylhydantoin, dibromoisocyanuric acid, monosodium bromoisocyanurate hydrate, boron tribromide, phosphorous tribromide, bromodimethylsulfonium bromide, 5,5-dibromomeldrum's acid, 2,4,4,6-tetrabromo-2,5-cyclohexadienone, bis(2,4,6-trimethylpyridine)-bromonium hexafluorophosphate, and trimethylsilyl bromide.

The term “borylating agent” refers to a chemical reagent that is used to add boron-containing functional groups to a, typically organic, reactant. Examples of “borylating agents” as used herein include, but are not limited to, tetrahydroxydiboron, catecholborane, 4,4,5,5-tetramethyl-1,3,2-dioxaborolane, 4,6,6-trimethyl-1,3,2-dioxaborinane, diisopropylamine borane, bis(neopentyl glycolato)diboron, bis(catecholato)diboron, bis(hexylene glycolato)diboron, bis(pinacolato)diboron, bis[(pinacolato) boryl]methane, 4-(4,4,5,5-tetramethyl-1,3,2-dioxaborolan-2-yl)-5-(trifluoromethyl)-1-(triisopropylsilyl)-1H-pyrrolo[2,3-b]pyridine, HB (trip) 2, 2-isopropoxy-4,4,5,5, -tetramethyl-1,3,2-dioxaborolane, 2-methoxy-4,4,5,5, -tetramethyl-1,3,2-dioxaborolane, and 2-ethoxy-4,4,5,5, -tetramethyl-1,3,2-dioxaborolane.

The term “acyl halide preparation agent” refers to a chemical reagent that is used to add an acyl halide functional group to a, typically organic, reactant. Examples of “acyl halide preparation agents” as used herein include, but are not limited to, oxalyl chloride, thionyl chloride, phosphoryl chloride, phosphorous trichloride, phosphorous pentachloride, phosgene, diphosgene, triphosgene, and cyanuric chloride.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In addition, compounds described herein include crystalline forms, also known as polymorphs. Polymorphs include the different crystal packing arrangements of the same elemental composition of a compound. Polymorphs usually have different X-ray diffraction patterns, melting points, density, hardness, crystal shape, optical properties, stability, and solubility. Various factors such as the recrystallization solvent, rate of crystallization, and storage temperature may cause a single crystal form to dominate.

The synthetic method disclosed herein is a method for producing CRAC channel inhibitors. In some embodiments, this method produces kilogram quantities. The methods may improve previous synthetic routes by eliminating the presence of multiple undesirable impurities.

### Improved Process of Synthesizing CRAC Channel Inhibitors

Provided herein is a process useful for preparing CRAC channel inhibitors and their intermediates. In particular, provided herein is a process and method for the manufacture of N-(5-(6-chloro-2,2-difluorobenzo[d][1,3]dioxol-5-yl) pyrazin-2-yl)-2-fluoro-6-methylbenzamide (Compound 3.1) or a pharmaceutically acceptable salt thereof, for example as shown in Scheme 1.

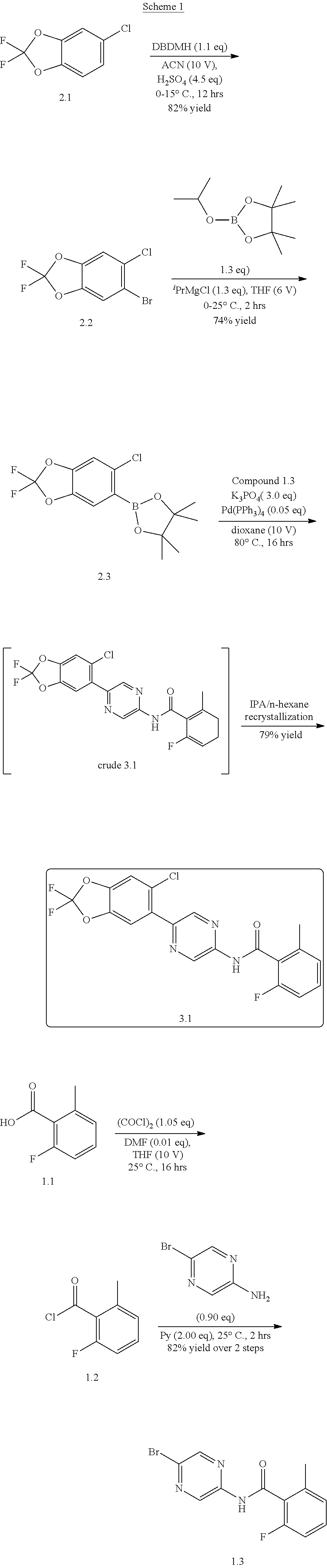

In some embodiments, the process offers improvements over previously disclosed processes. In some embodiments, the process of the present disclosure as shown in Scheme 1 offers improvements over the process disclosed in PCT/US2020/031506, as shown in Scheme 2.

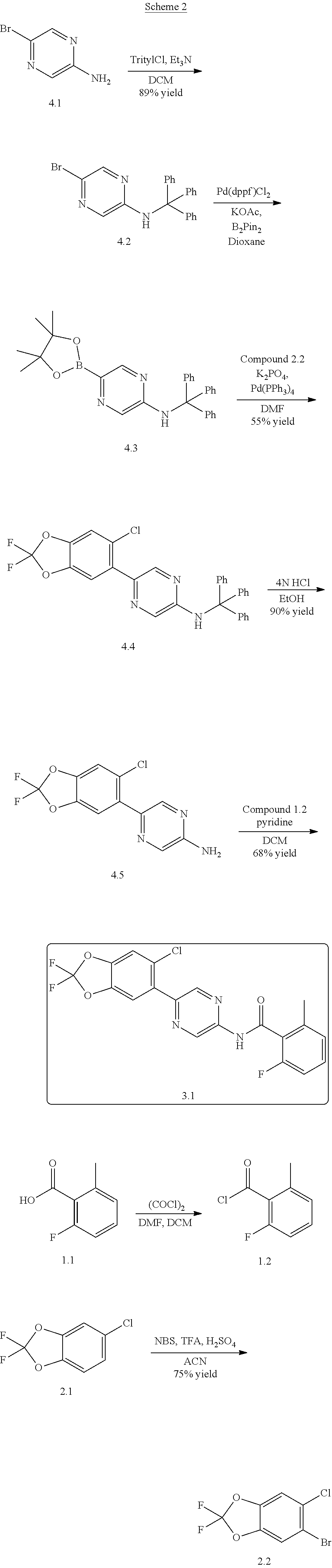

The process of the present disclosure provides Compound 3.1 from Compounds 1.1 and 2.1 in six synthetic steps, as opposed to seven synthetic steps for the process of PCT/US2020/031506. The lower number of overall steps results in lower usage of solvent and minimized waste and environmental impact. In particular, the process of the present disclosure avoids a number of solvents and reagents of concern, such as dichloromethane.

In the process of PCT/US2020/031506, the preparation of Compound 2.2 from Compound 2.1 suffered from overbromination to provide di-brominated impurities. Accordingly, the process provided Compound 2.2 in 75% yield and 87% purity. Conversely, the process of the present disclosure provides Compound 2.2 in 82% yield and 97% purity and avoids the generation of di-brominated impurities. Further, the current process is complete in 12 hours as opposed to the reaction time of 3 days of the process of PCT/US2020/031506.

In the process of PCT/US2020/031506, the preparation of borylated Suzuki coupling partner Compound 4.3 required a wasteful protection and deprotection scheme and resulted in the generation of a dehalogenated impurity. Conversely, the process of the present disclosure delivers borylated Suzuki coupling partner Compound 2.3 while avoiding the generation of dehalogenated impurities. This improvement greatly simplified the purification of Compound 3.1, resulting in the obtention of Compound 3.1 in quantitative purity.

Furthermore, the process of the present disclosure avoids the use of highly undesirable reagents such as trifluoroacetic acid, further minimizing the environmental impact of the process of the present disclosure. Additionally, the process of the present disclosure does not require column chromatography and hence avoids the use of silica gel.

In some embodiments, the processes described herein provide Compound 3.1 in higher overall yield (e.g., 45% overall yield as compared with 33% overall yield for the process of PCT/US2020/031506).

Provided herein, in one aspect, is a process of synthesizing CRAC channel inhibitors of Formula (I):

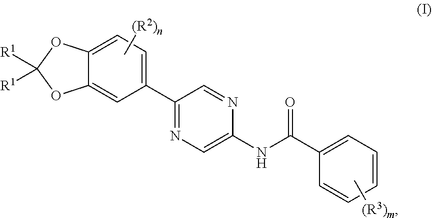

or a pharmaceutically acceptable salt thereof, wherein:
R¹, R², and R³ are independently selected at each occurrence from hydrogen, halogen, and C₁-C₃ alkyl optionally substituted with one or more substituents independently selected from hydrogen, halogen, —OH, —OR⁴, —CN, —N(R⁴)₂, and —NO₂;
or two R¹ groups are taken together with the atoms to which they are attached to form a carbocycle;
n is 0, 1, 2, or 3;
m is 0, 1, 2, 3, 4, or 5; and
R⁴ is independently selected at each occurrence from hydrogen; and C₁₋₆ alkyl, C₂₋₆ alkenyl, and C₂₋₆ alkynyl, each of which may be optionally substituted at each occurrence by halogen, —CN, —NO₂, —OH, —NH₂, and OCH₃.

Provided herein, in another aspect, is a process of synthesizing CRAC channel inhibitors of Formulas (IA), (IB), (IC), (ID), (IE), (IF), and (IG):

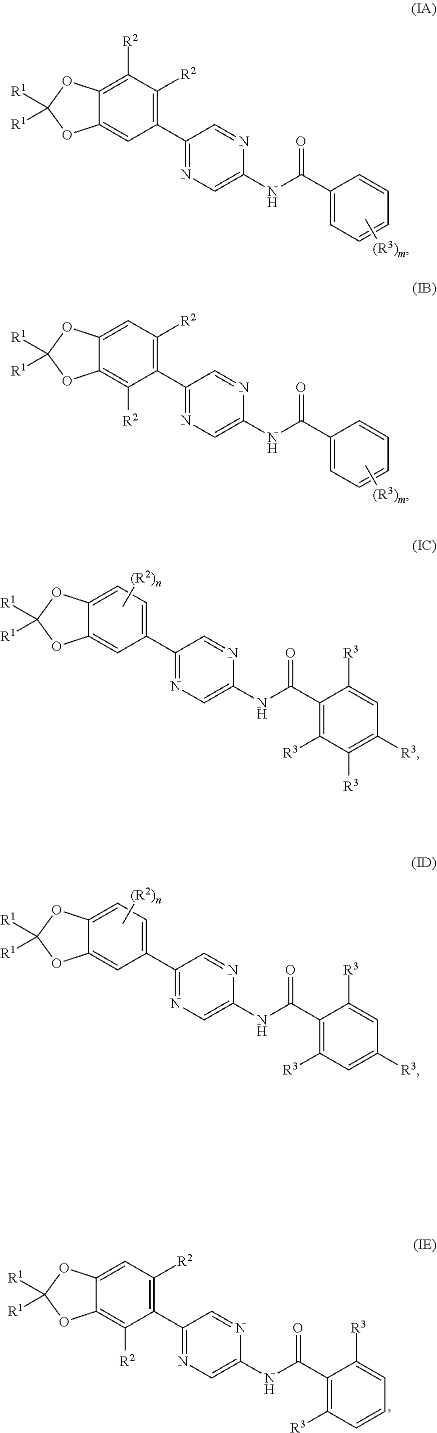

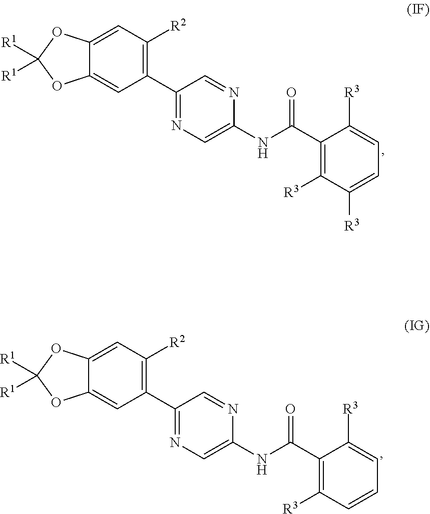

or a salt of any one thereof.

In certain embodiments, for a compound or salt of any one of Formulas (I), (IA), (IB), (IC), (ID), (IE), (IF), and (IG), R¹, R², and R³ are independently selected at each occurrence from hydrogen, halogen, and C₁-C₃ alkyl optionally substituted with one or more substituents independently selected from hydrogen, halogen, —OH, —OR⁴, —CN, —N(R⁴)₂, and —NO₂.

In certain embodiments, for a compound or salt of any one of Formulas (I), (IA), (IB), (IC), and (ID), n is 0, 1, 2, or 3. In certain embodiments, for a compound or salt any one of Formulas (I), (IA), (IB), (IC), and (ID), n is 0, 1, or 2. In certain embodiments, for a compound or salt any one of Formulas (I), (IA), (IB), (IC), and (ID), n is 0 or 1. In certain embodiments, for a compound or salt any one of Formulas (I), (IA), (IB), (IC), and (ID), nis 1.

In certain embodiments, for a compound or salt of any one of Formulas (I), (IA), and (IB), m is 0, 1, 2, 3, or 4. In certain embodiments, for a compound or salt of any one of Formulas (I), (IA), and (IB), m is 0, 1, 2, or 3. In certain embodiments, for a compound or salt of any one of Formulas (I), (IA), and (IB), m is 0, 1, or 2. In certain embodiments, for a compound or salt of any one of Formulas (I), (IA), and (IB), m is 2.

In certain embodiments, for a compound or salt of any one of Formulas (I), (IA), (IB), (IC), (ID), (IE), (IF), and (IG), R⁴ is independently selected at each occurrence from hydrogen; and C₁₋₆ alkyl, C₂₋₆ alkenyl, and C₂₋₆ alkynyl, each of which may be optionally substituted at each occurrence by halogen, —CN, —NO₂, —OH, —NH₂, and —OCH₃.

Provided herein, in another aspect, is a process of synthesizing CRAC channel inhibitors of Formulas (I), (IA), (IB), (IC), (ID), (IE), (IF), and (IG):

wherein the process comprises reacting a compound of Formula (I-A)

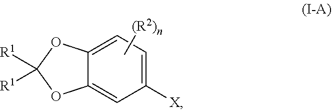

with a compound of Formula (I-B)

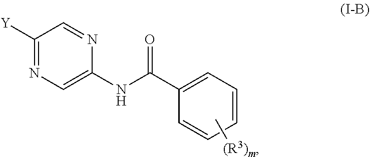

in the presence of a base, a catalyst, and a solvent, wherein:
X is selected from —B(OH)₂, —BF₃K,

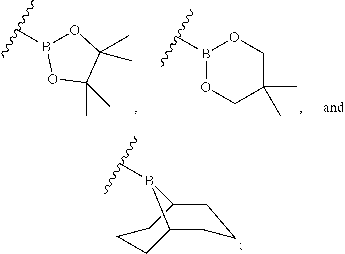

and
Y is selected from Cl, Br, and I.

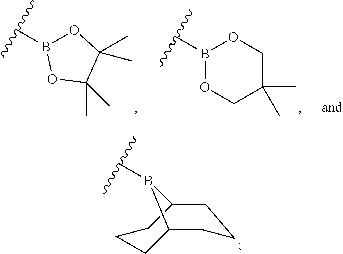

In some embodiments, X is selected from —B(OH)₂, —BF₃K,

In some embodiments, X is —B(OH)₂. In some embodiments, X is —BF₃K. In some embodiments, X is

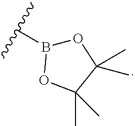 [SMILES: *B1OC(C)(C)C(C)(C)O1]

In some embodiments, X is

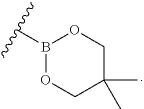 [SMILES: *B1OCC(C)(C)CO1]

In some embodiments, X is

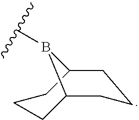 [SMILES: *B1C2CCCC1CCC2]

In some embodiments, Y is selected from Cl, Br, and I. In some embodiments, Y is Cl. In some embodiments, Y is Br. In some embodiments, Y is I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium carbonate. In some embodiments, the base is sodium carbonate. In some embodiments, the base is potassium bicarbonate. In some embodiments, the base is sodium bicarbonate. In some embodiments, the base is piperidine. In some embodiments, the base is pyridine. In some embodiments, the base is 1,8-diazabicyclo[5.4.0]undec-7-ene. In some embodiments, the base is sodium tert-butoxide. In some embodiments, the base is potassium tert-butoxide. In some embodiments, the base is cesium carbonate. In some embodiments, the base is potassium phosphate. In some embodiments, the base is sodium hydroxide. In some embodiments, the base is N,N-diisopropylethylamine. In some embodiments, the base is triethylamine.

In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄(BF₄)₂, PdCl₂, XPhos-Pd-G3, Pd-PEPPSI™—IPr, Pd—PEPPSI™—SIPr, and Pd-PEPPSI™-IPent. In some embodiments, the catalyst is Pd(acac)₂. In some embodiments, the catalyst is [Pd(allyl)Cl]₂. In some embodiments, the catalyst is Pd(MeCN)₂Cl₂. In some embodiments, the catalyst is Pd(dba)₂. In some embodiments, the catalyst is Pd(TFA)₂. In some embodiments, the catalyst is Pd₂(dba)₃. In some embodiments, the catalyst is Pd₂(dba)₃·CHCl₃. In some embodiments, the catalyst is Pd(PPh₃)₄. In some embodiments, the catalyst is Pd(OAc)₂. In some embodiments, the catalyst is Pd(PCy₃)₂Cl₂. In some embodiments, the catalyst is Pd(PPh₃)₂Cl₂. In some embodiments, the catalyst is Pd[P(o-tol)₃]₂Cl₂. In some embodiments, the catalyst is Pd(amphos)Cl₂. In some embodiments, the catalyst is Pd(dppf)Cl₂. In some embodiments, the catalyst is Pd(dppf)Cl₂·CH₂Cl₂. In some embodiments, the catalyst is Pd(dtbpf)Cl₂. In some embodiments, the catalyst is Pd(MeCN)₄(BF₄)₂. In some embodiments, the catalyst is PdCl₂. In some embodiments, the catalyst is XPhos-Pd-G3. In some embodiments, the catalyst is Pd-PEPPSI™—IPr. In some embodiments, the catalyst is Pd-PEPPSI™—SIPr. In some embodiments, the catalyst is Pd-PEPPSI™-IPent.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (I-A), the compound of Formula (I-B), the base, the catalyst, and the solvent are stirred:
for no longer than 16 hours; and
at a temperature of between about 75° C. and about 80° C.

In some embodiments, the process further comprises precipitating the compound of Formula (I) and isolating it by filtration.

In some embodiments, the process provides the compound of Formula (I) in a synthetic yield of greater than about 60%. In some embodiments, the process provides the compound of Formula (I) in a synthetic yield of greater than about 65%. In some embodiments, the process provides the compound of Formula (I) in a synthetic yield of greater than about 70%. In some embodiments, the process provides the compound of Formula (I) in a synthetic yield of greater than about 75%. In some embodiments, the process provides the compound of Formula (I) in a synthetic yield of greater than about 80%.

In some embodiments, the compound of Formula (I-A)

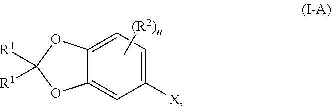

is synthesized by reacting a compound of Formula (I-C)

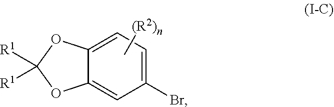

with a borylating agent in the presence of a base and a solvent.

In some embodiments, the borylating agent is selected from 2-isopropoxy-4,4,5,5, -tetramethyl-1,3,2-dioxaborolane, 2-methoxy-4,4,5,5, -tetramethyl-1,3,2-dioxaborolane, and 2-ethoxy-4,4,5,5, -tetramethyl-1,3,2-dioxaborolane. In some embodiments, the borylating agent is 2-isopropoxy-4,4,5,5, -tetramethyl-1,3,2-dioxaborolane. In some embodiments, the borylating agent is 2-methoxy-4,4,5,5, -tetramethyl-1,3,2-dioxaborolane. In some embodiments, the borylating agent is 2-ethoxy-4,4,5,5, -tetramethyl-1,3,2-dioxaborolane.

In some embodiments, the base is selected from isopropylmagnesium chloride, isopropylmagnesium lithium chloride, methylmagnesium bromide, methylmagnesium chloride, methylmagnesium iodide, ethylmagnesium chloride, ethylmagnesium bromide, isopropylmagnesium bromide, methyllithium, ethyllithium, isopropyllithium, n-butyllithium, and tert-butyllithium. In some embodiments, the base is isopropylmagnesium chloride. In some embodiments, the base is isopropylmagnesium lithium chloride. In some embodiments, the base is methylmagnesium bromide. In some embodiments, the base is methylmagnesium chloride. In some embodiments, the base is methylmagnesium iodide. In some embodiments, the base is ethylmagnesium chloride. In some embodiments, the base is ethylmagnesium bromide. In some embodiments, the base is isopropylmagnesium bromide. In some embodiments, the base is methyllithium. In some embodiments, the base is ethyllithium. In some embodiments, the base is isopropyllithium. In some embodiments, the base is n-butyllithium. In some embodiments, the base is tert-butyllithium.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (I-C), the borylating agent, the base, and the solvent are stirred:
for no longer than 2 hours; and
at a temperature of between about 0° C. and about 25° C.

In some embodiments, the process further comprises precipitating the compound of Formula (I-A) and isolating it by filtration.

In some embodiments, the process provides the compound of Formula (I-A) in a synthetic yield of greater than about 60%. In some embodiments, the process provides the compound of Formula (I-A) in a synthetic yield of greater than about 65%. In some embodiments, the process provides the compound of Formula (I-A) in a synthetic yield of greater than about 70%. In some embodiments, the process provides the compound of Formula (I-A) in a synthetic yield of greater than about 75%.

In some embodiments, the compound of Formula (I-C)

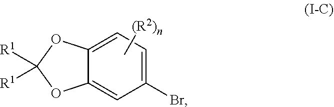

is synthesized by reacting a compound of Formula (I-D)

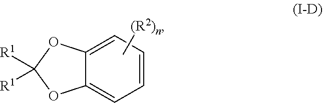

with a brominating agent in the presence of an acid and a solvent.

In some embodiments, the brominating agent is selected from N-bromosuccinimide, tribromoisocyanuric acid, 1,3-dibromo-5,5-dimethylhydantoin, and bromine. In some embodiments, the brominating agent is N-bromosuccinimide. In some embodiments, the brominating agent is tribromoisocyanuric acid. In some embodiments, the brominating agent is 1,3-dibromo-5,5-dimethylhydantoin. In some embodiments, the brominating agent is bromine.

In some embodiments, the acid is selected from hydrochloric acid, sulfuric acid, nitric acid, acetic acid, hydrobromic acid, phosphoric acid, formic acid, and trifluoroacetic acid. In some embodiments, the acid is hydrochloric acid. In some embodiments, the acid is sulfuric acid. In some embodiments, the acid is nitric acid. In some embodiments, the acid is acetic acid. In some embodiments, the acid is hydrobromic acid. In some embodiments, the acid is phosphoric acid. In some embodiments, the acid is formic acid. In some embodiments, the acid is trifluoroacetic acid.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (I-D), the brominating agent, the acid, and the solvent are stirred:
for no longer than 12 hours; and
at a temperature of between about 0° C. and about 15° C.

In some embodiments, the process further comprises extracting the compound of Formula (I-C) and isolating it by concentration.

In some embodiments, the process provides the compound of Formula (I-C) in a synthetic yield of greater than about 70%. In some embodiments, the process provides the compound of Formula (I-C) in a synthetic yield of greater than about 75%. In some embodiments, the process provides the compound of Formula (I-C) in a synthetic yield of greater than about 80%. In some embodiments, the process provides the compound of Formula (I-C) in a synthetic yield of greater than about 85%.

In some embodiments, the compound of Formula (I-B)

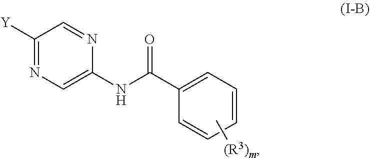

is synthesized by reacting a compound of Formula (I-E)

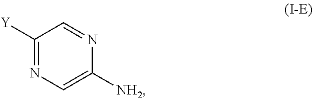

with a compound of Formula (I-F)

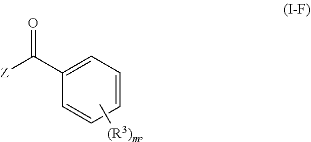

in the presence of a base, wherein:
Z is selected from Cl, Br, and I.

In some embodiments, Z is selected from Cl, Br, and I. In some embodiments, Z is Cl. In some embodiments, Z is Br. In some embodiments, Z is I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium carbonate. In some embodiments, the base is sodium carbonate. In some embodiments, the base is potassium bicarbonate. In some embodiments, the base is sodium bicarbonate. In some embodiments, the base is piperidine. In some embodiments, the base is pyridine. In some embodiments, the base is 1,8-diazabicyclo[5.4.0]undec-7-ene. In some embodiments, the base is sodium tert-butoxide. In some embodiments, the base is potassium tert-butoxide. In some embodiments, the base is cesium carbonate. In some embodiments, the base is potassium phosphate. In some embodiments, the base is sodium hydroxide. In some embodiments, the base is N,N-diisopropylethylamine. In some embodiments, the base is triethylamine.

In some embodiments, the compound of Formula (I-E), the compound of Formula (I-F), and the base are stirred:
for no longer than 2 hours; and
at a temperature of between about 20° C. and about 25° C.

In some embodiments, the process further comprises precipitating the compound of Formula (I-B) and isolating it by filtration.

In some embodiments, the process provides the compound of Formula (I-B) in a synthetic yield of greater than about 70%. In some embodiments, the process provides the compound of Formula (I-B) in a synthetic yield of greater than about 75%. In some embodiments, the process provides the compound of Formula (I-B) in a synthetic yield of greater than about 80%. In some embodiments, the process provides the compound of Formula (I-B) in a synthetic yield of greater than about 85%.

In some embodiments, the compound of Formula (I-F)

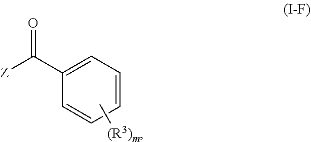

is synthesized by reacting a compound of Formula (I-G)

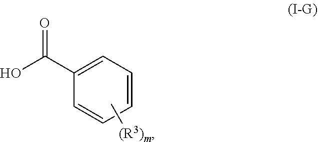

with an acyl halide preparation agent in the presence of a solvent.

In some embodiments, the acyl halide preparation agent is selected from oxalyl chloride, thionyl chloride, phosphoryl chloride, phosphorous trichloride, phosphorous pentachloride, phosgene, diphosgene, triphosgene, and cyanuric chloride. In some embodiments, the acyl halide preparation agent is oxalyl chloride. In some embodiments, the acyl halide preparation agent is thionyl chloride. In some embodiments, the acyl halide preparation agent is phosphoryl chloride. In some embodiments, the acyl halide preparation agent is phosphorous trichloride. In some embodiments, the acyl halide preparation agent is phosphorous pentachloride. In some embodiments, the acyl halide preparation agent is phosgene. In some embodiments, the acyl halide preparation agent is diphosgene. In some embodiments, the acyl halide preparation agent is triphosgene. In some embodiments, the acyl halide preparation agent is cyanuric chloride.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (I-G), the acyl halide preparation agent, and the solvent are stirred:
for no longer than 16 hours; and
at a temperature of between about 20° C. and about 25° C.

Provided herein, in one aspect, is a process of synthesizing CRAC channel inhibitors of Formula (IE):

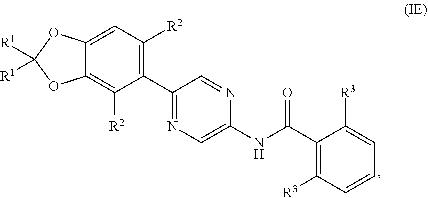

or a pharmaceutically acceptable salt thereof, wherein:
R¹, R², and R³ are independently selected at each occurrence from hydrogen, halogen, and C₁-C₃ alkyl optionally substituted with one or more substituents independently selected from hydrogen, halogen, —OH, —OR⁴, —CN, —N(R⁴)₂, and —NO₂;
or two R¹ groups are taken together with the atoms to which they are attached to form a carbocycle; and
R⁴ is independently selected at each occurrence from hydrogen; and C₁₋₆ alkyl, C₂₋₆ alkenyl, and C₂₋₆ alkynyl, each of which may be optionally substituted at each occurrence by halogen, —CN, —NO₂, —OH, —NH₂, and OCH₃;
   wherein the process comprises reacting a compound of Formula (IE-A)

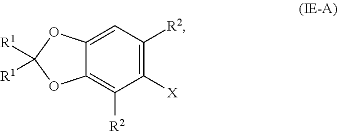

with a compound of Formula (IE-B)

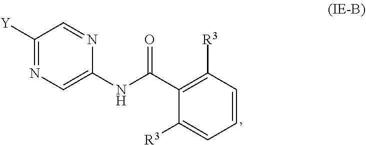

in the presence of a base, a catalyst, and a solvent, wherein:
X is selected from —B(OH)₂, —BF₃K,

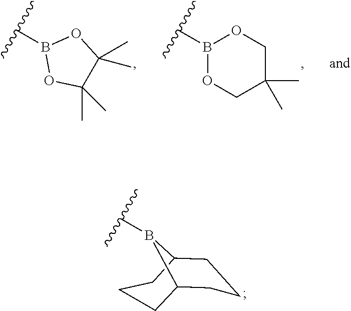

and
Y is selected from Cl, Br, and I.

In some embodiments, X is selected from —B(OH)₂, —BF₃K,

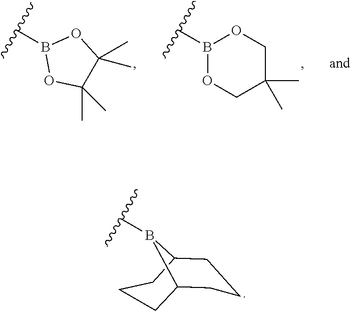

In some embodiments, X is —B(OH) 2. In some embodiments, X is —BF₃K. In some embodiments, X is

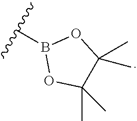 [SMILES: *B1OC(C)(C)C(C)(C)O1]

In some embodiments, X is

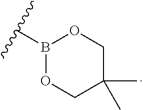 [SMILES: *B1OCC(C)(C)CO1]

In some embodiments, X is

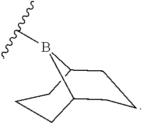 [SMILES: *B1C2CCCC1CCC2]

In some embodiments, Y is selected from Cl, Br, and I. In some embodiments, Y is Cl. In some embodiments, Y is Br. In some embodiments, Y is I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium carbonate. In some embodiments, the base is sodium carbonate. In some embodiments, the base is potassium bicarbonate. In some embodiments, the base is sodium bicarbonate. In some embodiments, the base is piperidine. In some embodiments, the base is pyridine. In some embodiments, the base is 1,8-diazabicyclo[5.4.0]undec-7-ene. In some embodiments, the base is sodium tert-butoxide. In some embodiments, the base is potassium tert-butoxide. In some embodiments, the base is cesium carbonate. In some embodiments, the base is potassium phosphate. In some embodiments, the base is sodium hydroxide. In some embodiments, the base is N,N-diisopropylethylamine. In some embodiments, the base is triethylamine.

In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄(BF₄)₂, PdCl₂, XPhos-Pd-G₃, Pd-PEPPSI™—IPr, Pd—PEPPSI™—SIPr, and Pd-PEPPSI™-IPent. In some embodiments, the catalyst is Pd(acac) 2. In some embodiments, the catalyst is [Pd(allyl)Cl]₂. In some embodiments, the catalyst is Pd(MeCN)₂Cl₂. In some embodiments, the catalyst is Pd(dba)₂. In some embodiments, the catalyst is Pd(TFA)₂. In some embodiments, the catalyst is Pd₂(dba)₃. In some embodiments, the catalyst is Pd₂(dba)₃·CHCl₃. In some embodiments, the catalyst is Pd(PPh₃)₄. In some embodiments, the catalyst is Pd(OAc)₂. In some embodiments, the catalyst is Pd(PCy₃)₂Cl₂. In some embodiments, the catalyst is Pd(PPh₃)₂Cl₂. In some embodiments, the catalyst is Pd[P(o-tol)₃]₂Cl₂. In some embodiments, the catalyst is Pd(amphos)Cl₂. In some embodiments, the catalyst is Pd(dppf)Cl₂. In some embodiments, the catalyst is Pd(dppf)Cl₂·CH₂Cl₂. In some embodiments, the catalyst is Pd(dtbpf)Cl₂. In some embodiments, the catalyst is Pd(MeCN)₄(BF₄)₂. In some embodiments, the catalyst is PdCl₂. In some embodiments, the catalyst is XPhos-Pd-G3. In some embodiments, the catalyst is Pd-PEPPSI™—IPr. In some embodiments, the catalyst is Pd-PEPPSI™—SIPr. In some embodiments, the catalyst is Pd-PEPPSI™-IPent.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (IE-A), the compound of Formula (IE-B), the base, the catalyst, and the solvent are stirred:
for no longer than 16 hours; and
at a temperature of between about 75° C. and about 80° C.

In some embodiments, the process further comprises precipitating the compound of Formula (IE) and isolating it by filtration.

In some embodiments, the process provides the compound of Formula (IE) in a synthetic yield of greater than about 60%. In some embodiments, the process provides the compound of Formula (IE) in a synthetic yield of greater than about 65%. In some embodiments, the process provides the compound of Formula (IE) in a synthetic yield of greater than about 70%. In some embodiments, the process provides the compound of Formula (IE) in a synthetic yield of greater than about 75%. In some embodiments, the process provides the compound of Formula (IE) in a synthetic yield of greater than about 80%.

In some embodiments, the compound of Formula (IE-A)

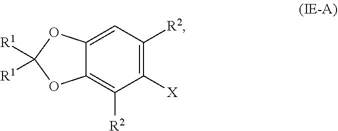

is synthesized by reacting a compound of Formula (IE-C)

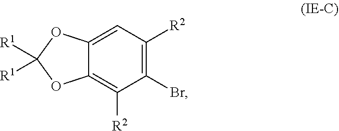

with a borylating agent in the presence of a base and a solvent.

In some embodiments, the borylating agent is selected from 2-isopropoxy-4,4,5,5, -tetramethyl-1,3,2-dioxaborolane, 2-methoxy-4,4,5,5, -tetramethyl-1,3,2-dioxaborolane, and 2-ethoxy-4,4,5,5, -tetramethyl-1,3,2-dioxaborolane. In some embodiments, the borylating agent is 2-isopropoxy-4,4,5,5, -tetramethyl-1,3,2-dioxaborolane. In some embodiments, the borylating agent is 2-methoxy-4,4,5,5, -tetramethyl-1,3,2-dioxaborolane. In some embodiments, the borylating agent is 2-ethoxy-4,4,5,5, -tetramethyl-1,3,2-dioxaborolane.

In some embodiments, the base is selected from isopropylmagnesium chloride, isopropylmagnesium lithium chloride, methylmagnesium bromide, methylmagnesium chloride, methylmagnesium iodide, ethylmagnesium chloride, ethylmagnesium bromide, isopropylmagnesium bromide, methyllithium, ethyllithium, isopropyllithium, n-butyllithium, and tert-butyllithium. In some embodiments, the base is isopropylmagnesium chloride. In some embodiments, the base is isopropylmagnesium lithium chloride. In some embodiments, the base is methylmagnesium bromide. In some embodiments, the base is methylmagnesium chloride. In some embodiments, the base is methylmagnesium iodide. In some embodiments, the base is ethylmagnesium chloride. In some embodiments, the base is ethylmagnesium bromide. In some embodiments, the base is isopropylmagnesium bromide. In some embodiments, the base is methyllithium. In some embodiments, the base is ethyllithium. In some embodiments, the base is isopropyllithium. In some embodiments, the base is n-butyllithium. In some embodiments, the base is tert-butyllithium.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (IE-C), the borylating agent, the base, and the solvent are stirred:
for no longer than 2 hours; and
at a temperature of between about 0° C. and about 25° C.

In some embodiments, the process further comprises precipitating the compound of Formula (IE-A) and isolating it by filtration.

In some embodiments, the process provides the compound of Formula (IE-A) in a synthetic yield of greater than about 60%. In some embodiments, the process provides the compound of Formula (IE-A) in a synthetic yield of greater than about 65%. In some embodiments, the process provides the compound of Formula (IE-A) in a synthetic yield of greater than about 70%. In some embodiments, the process provides the compound of Formula (IE-A) in a synthetic yield of greater than about 75%.

In some embodiments, the compound of Formula (IE-C)

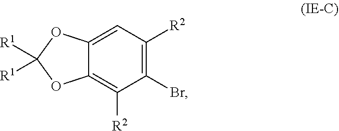

is synthesized by reacting a compound of Formula (IE-D)

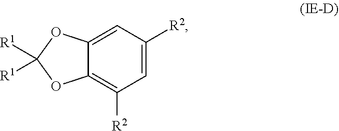

with a brominating agent in the presence of an acid and a solvent.

In some embodiments, the brominating agent is selected from N-bromosuccinimide, tribromoisocyanuric acid, 1,3-dibromo-5,5-dimethylhydantoin, and bromine. In some embodiments, the brominating agent is N-bromosuccinimide. In some embodiments, the brominating agent is tribromoisocyanuric acid. In some embodiments, the brominating agent is 1,3-dibromo-5,5-dimethylhydantoin. In some embodiments, the brominating agent is bromine.

In some embodiments, the acid is selected from hydrochloric acid, sulfuric acid, nitric acid, acetic acid, hydrobromic acid, phosphoric acid, formic acid, and trifluoroacetic acid. In some embodiments, the acid is hydrochloric acid. In some embodiments, the acid is sulfuric acid. In some embodiments, the acid is nitric acid. In some embodiments, the acid is acetic acid. In some embodiments, the acid is hydrobromic acid. In some embodiments, the acid is phosphoric acid. In some embodiments, the acid is formic acid. In some embodiments, the acid is trifluoroacetic acid.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (IE-D), the brominating agent, the acid, and the solvent are stirred:
for no longer than 12 hours; and
at a temperature of between about 0° C. and about 15° C.

In some embodiments, the process further comprises extracting the compound of Formula (IE-C) and isolating it by concentration.

In some embodiments, the process provides the compound of Formula (IE-C) in a synthetic yield of greater than about 70%. In some embodiments, the process provides the compound of Formula (IE-C) in a synthetic yield of greater than about 75%. In some embodiments, the process provides the compound of Formula (IE-C) in a synthetic yield of greater than about 80%. In some embodiments, the process provides the compound of Formula (IE-C) in a synthetic yield of greater than about 85%.

In some embodiments, the compound of Formula (IE-B)

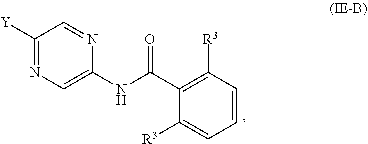

is synthesized by reacting a compound of Formula (I-E)

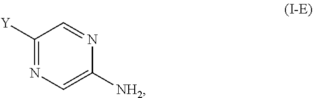

with a compound of Formula (IE-F)

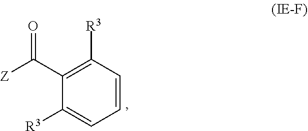

in the presence of a base, wherein:
Z is selected from Cl, Br, and I.

In some embodiments, Z is selected from Cl, Br, and I. In some embodiments, Z is Cl. In some embodiments, Z is Br. In some embodiments, Z is I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium carbonate. In some embodiments, the base is sodium carbonate. In some embodiments, the base is potassium bicarbonate. In some embodiments, the base is sodium bicarbonate. In some embodiments, the base is piperidine. In some embodiments, the base is pyridine. In some embodiments, the base is 1,8-diazabicyclo[5.4.0]undec-7-ene. In some embodiments, the base is sodium tert-butoxide. In some embodiments, the base is potassium tert-butoxide. In some embodiments, the base is cesium carbonate. In some embodiments, the base is potassium phosphate. In some embodiments, the base is sodium hydroxide. In some embodiments, the base is N,N-diisopropylethylamine. In some embodiments, the base is triethylamine.

In some embodiments, the compound of Formula (I-E), the compound of Formula (IE-F), and the base are stirred:
for no longer than 2 hours; and
at a temperature of between about 20° C. and about 25° C.

In some embodiments, the process further comprises precipitating the compound of Formula (IE-B) and isolating it by filtration.

In some embodiments, the process provides the compound of Formula (IE-B) in a synthetic yield of greater than about 70%. In some embodiments, the process provides the compound of Formula (IE-B) in a synthetic yield of greater than about 75%. In some embodiments, the process provides the compound of Formula (IE-B) in a synthetic yield of greater than about 80%. In some embodiments, the process provides the compound of Formula (IE-B) in a synthetic yield of greater than about 85%.

In some embodiments, the compound of Formula (IE-F)

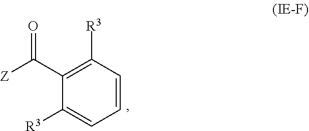

is synthesized by reacting a compound of Formula (IE-G)

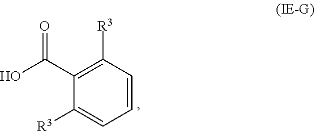

with an acyl halide preparation agent in the presence of a solvent.

In some embodiments, the acyl halide preparation agent is selected from oxalyl chloride, thionyl chloride, phosphoryl chloride, phosphorous trichloride, phosphorous pentachloride, phosgene, diphosgene, triphosgene, and cyanuric chloride. In some embodiments, the acyl halide preparation agent is oxalyl chloride. In some embodiments, the acyl halide preparation agent is thionyl chloride. In some embodiments, the acyl halide preparation agent is phosphoryl chloride. In some embodiments, the acyl halide preparation agent is phosphorous trichloride. In some embodiments, the acyl halide preparation agent is phosphorous pentachloride. In some embodiments, the acyl halide preparation agent is phosgene. In some embodiments, the acyl halide preparation agent is diphosgene. In some embodiments, the acyl halide preparation agent is triphosgene. In some embodiments, the acyl halide preparation agent is cyanuric chloride.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (IE-G), the acyl halide preparation agent, and the solvent are stirred:
for no longer than 16 hours; and
at a temperature of between about 20° C. and about 25° C.

Provided herein, in another aspect, is a process of synthesizing CRAC channel inhibitors of Formula (II):

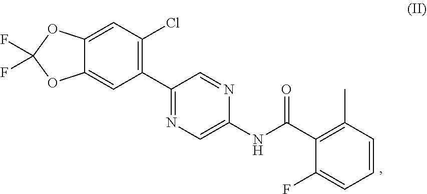 [SMILES: Cc1cccc(F)c1C(=O)Nc1cnc(-c2cc3c(cc2Cl)OC(F)(F)O3)cn1]

or a pharmaceutically acceptable salt thereof, wherein the process comprises reacting a compound of Formula (II-A)

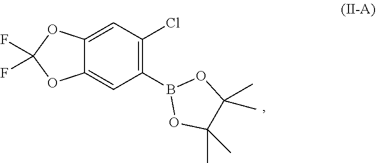 [SMILES: CC1(C)OB(c2cc3c(cc2Cl)OC(F)(F)O3)OC1(C)C]

with a compound of Formula (I-B)

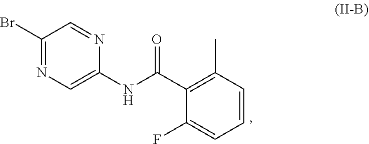 [SMILES: Cc1cccc(F)c1C(=O)Nc1cnc(Br)cn1]

in the presence of a base, a catalyst, and a solvent.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium carbonate. In some embodiments, the base is sodium carbonate. In some embodiments, the base is potassium bicarbonate. In some embodiments, the base is sodium bicarbonate. In some embodiments, the base is piperidine. In some embodiments, the base is pyridine. In some embodiments, the base is 1,8-diazabicyclo[5.4.0]undec-7-ene. In some embodiments, the base is sodium tert-butoxide. In some embodiments, the base is potassium tert-butoxide. In some embodiments, the base is cesium carbonate. In some embodiments, the base is potassium phosphate. In some embodiments, the base is sodium hydroxide. In some embodiments, the base is N,N-diisopropylethylamine. In some embodiments, the base is triethylamine.

In some embodiments, the catalyst is selected from Pd(acac)₂, [Pd(allyl)Cl]₂, Pd (MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄(BF₄)₂, PdCl₂, XPhos-Pd-G3, Pd-PEPPSI™—IPr, Pd—PEPPSI™—SIPr, and Pd-PEPPSI™-IPent. In some embodiments, the catalyst is Pd(acac)₂. In some embodiments, the catalyst is [Pd(allyl)Cl]₂. In some embodiments, the catalyst is Pd(MeCN)₂Cl₂. In some embodiments, the catalyst is Pd(dba)₂. In some embodiments, the catalyst is Pd(TFA)₂. In some embodiments, the catalyst is Pd₂(dba)₃. In some embodiments, the catalyst is Pd₂(dba)₃·CHCl₃. In some embodiments, the catalyst is Pd(PPh₃)₄. In some embodiments, the catalyst is Pd(OAc)₂. In some embodiments, the catalyst is Pd(PCy₃)₂Cl₂. In some embodiments, the catalyst is Pd(PPh₃)₂Cl₂. In some embodiments, the catalyst is Pd[P(o-tol)₃]₂Cl₂. In some embodiments, the catalyst is Pd(amphos)Cl₂. In some embodiments, the catalyst is Pd(dppf)Cl₂. In some embodiments, the catalyst is Pd(dppf)Cl₂·CH₂Cl₂. In some embodiments, the catalyst is Pd(dtbpf)Cl₂. In some embodiments, the catalyst is Pd(MeCN)₄(BF₄)₂. In some embodiments, the catalyst is PdCl₂. In some embodiments, the catalyst is XPhos-Pd-G3. In some embodiments, the catalyst is Pd-PEPPSI™—IPr. In some embodiments, the catalyst is Pd-PEPPSI™—SIPr. In some embodiments, the catalyst is Pd-PEPPSI™-IPent.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (II-A), the compound of Formula (II-B), the base, the catalyst, and the solvent are stirred:
for no longer than 16 hours; and
at a temperature of between about 75° C. and about 80° C.

In some embodiments, the process further comprises precipitating the compound of Formula (II) and isolating it by filtration.

In some embodiments, the process provides the compound of Formula (II) in a synthetic yield of greater than about 60%. In some embodiments, the process provides the compound of Formula (II) in a synthetic yield of greater than about 65%. In some embodiments, the process provides the compound of Formula (II) in a synthetic yield of greater than about 70%. In some embodiments, the process provides the compound of Formula (II) in a synthetic yield of greater than about 75%. In some embodiments, the process provides the compound of Formula (I) in a synthetic yield of greater than about 80%.

In some embodiments, the compound of Formula (II-A)

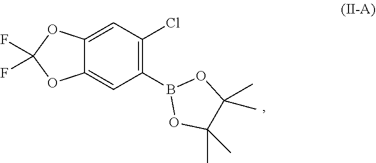 [SMILES: CC1(C)OB(c2cc3c(cc2Cl)OC(F)(F)O3)OC1(C)C]

is synthesized by reacting a compound of Formula (II-C)

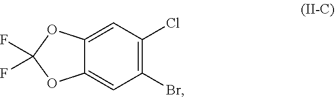 [SMILES: FC1(F)Oc2cc(Cl)c(Br)cc2O1]

with a borylating agent in the presence of a base and a solvent.

In some embodiments, the borylating agent is selected from 2-isopropoxy-4,4,5,5, -tetramethyl-1,3,2-dioxaborolane, 2-methoxy-4,4,5,5, -tetramethyl-1,3,2-dioxaborolane, and 2-ethoxy-4,4,5,5, -tetramethyl-1,3,2-dioxaborolane. In some embodiments, the borylating agent is 2-isopropoxy-4,4,5,5, -tetramethyl-1,3,2-dioxaborolane. In some embodiments, the borylating agent is 2-methoxy-4,4,5,5, -tetramethyl-1,3,2-dioxaborolane. In some embodiments, the borylating agent is 2-ethoxy-4,4,5,5, -tetramethyl-1,3,2-dioxaborolane.

In some embodiments, the base is selected from isopropylmagnesium chloride, isopropylmagnesium lithium chloride, methylmagnesium bromide, methylmagnesium chloride, methylmagnesium iodide, ethylmagnesium chloride, ethylmagnesium bromide, isopropylmagnesium bromide, methyllithium, ethyllithium, isopropyllithium, n-butyllithium, and tert-butyllithium. In some embodiments, the base is isopropylmagnesium chloride. In some embodiments, the base is isopropylmagnesium lithium chloride. In some embodiments, the base is methylmagnesium bromide. In some embodiments, the base is methylmagnesium chloride. In some embodiments, the base is methylmagnesium iodide. In some embodiments, the base is ethylmagnesium chloride. In some embodiments, the base is ethylmagnesium bromide. In some embodiments, the base is isopropylmagnesium bromide. In some embodiments, the base is methyllithium. In some embodiments, the base is ethyllithium. In some embodiments, the base is isopropyllithium. In some embodiments, the base is n-butyllithium. In some embodiments, the base is tert-butyllithium.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (II-C), the borylating agent, the base, and the solvent are stirred:
for no longer than 2 hours; and
at a temperature of between about 0° C. and about 25° C.

In some embodiments, the process further comprises precipitating the compound of Formula (II-A) and isolating it by filtration.

In some embodiments, the process provides the compound of Formula (II-A) in a synthetic yield of greater than about 60%. In some embodiments, the process provides the compound of Formula (II-A) in a synthetic yield of greater than about 65%. In some embodiments, the process provides the compound of Formula (II-A) in a synthetic yield of greater than about 70%. In some embodiments, the process provides the compound of Formula (II-A) in a synthetic yield of greater than about 75%.

In some embodiments, the compound of Formula (II-C)

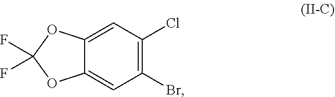 [SMILES: FC1(F)Oc2cc(Cl)c(Br)cc2O1]

is synthesized by reacting a compound of Formula (II-D)

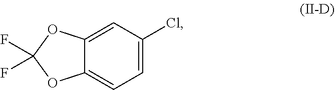 [SMILES: FC1(F)Oc2ccc(Cl)cc2O1]

with a brominating agent in the presence of an acid and a solvent.

In some embodiments, the brominating agent is selected from N-bromosuccinimide, tribromoisocyanuric acid, 1,3-dibromo-5,5-dimethylhydantoin, and bromine. In some embodiments, the brominating agent is N-bromosuccinimide. In some embodiments, the brominating agent is tribromoisocyanuric acid. In some embodiments, the brominating agent is 1,3-dibromo-5,5-dimethylhydantoin. In some embodiments, the brominating agent is bromine.

In some embodiments, the acid is selected from hydrochloric acid, sulfuric acid, nitric acid, acetic acid, hydrobromic acid, phosphoric acid, formic acid, and trifluoroacetic acid. In some embodiments, the acid is hydrochloric acid. In some embodiments, the acid is sulfuric acid. In some embodiments, the acid is nitric acid. In some embodiments, the acid is acetic acid. In some embodiments, the acid is hydrobromic acid. In some embodiments, the acid is phosphoric acid. In some embodiments, the acid is formic acid. In some embodiments, the acid is trifluoroacetic acid.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (II-D), the brominating agent, the acid, and the solvent are stirred:
for no longer than 12 hours; and
at a temperature of between about 0° C. and about 15° C.

In some embodiments, the process further comprises extracting the compound of Formula (II-C) and isolating it by concentration.

In some embodiments, the process provides the compound of Formula (II-C) in a synthetic yield of greater than about 70%. In some embodiments, the process provides the compound of Formula (II-C) in a synthetic yield of greater than about 75%. In some embodiments, the process provides the compound of Formula (II-C) in a synthetic yield of greater than about 80%. In some embodiments, the process provides the compound of Formula (II-C) in a synthetic yield of greater than about 85%.

In some embodiments, the compound of Formula (II-B)

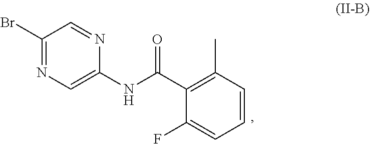 [SMILES: Cc1cccc(F)c1C(=O)Nc1cnc(Br)cn1]

is synthesized by reacting a compound of Formula (II-E)

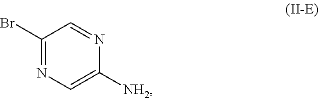

with a compound of Formula (II-F)

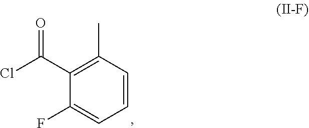

in the presence of a base, wherein:
Z is selected from Cl, Br, and I.

In some embodiments, the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine. In some embodiments, the base is potassium carbonate. In some embodiments, the base is sodium carbonate. In some embodiments, the base is potassium bicarbonate. In some embodiments, the base is sodium bicarbonate. In some embodiments, the base is piperidine. In some embodiments, the base is pyridine. In some embodiments, the base is 1,8-diazabicyclo[5.4.0]undec-7-ene. In some embodiments, the base is sodium tert-butoxide. In some embodiments, the base is potassium tert-butoxide. In some embodiments, the base is cesium carbonate. In some embodiments, the base is potassium phosphate. In some embodiments, the base is sodium hydroxide. In some embodiments, the base is N,N-diisopropylethylamine. In some embodiments, the base is triethylamine.

In some embodiments, the compound of Formula (II-E), the compound of Formula (II-F), and the base are stirred:
for no longer than 2 hours; and
at a temperature of between about 20° C. and about 25° C.

In some embodiments, the process further comprises precipitating the compound of Formula (II-B) and isolating it by filtration.

In some embodiments, the process provides the compound of Formula (II-B) in a synthetic yield of greater than about 70%. In some embodiments, the process provides the compound of Formula (II-B) in a synthetic yield of greater than about 75%. In some embodiments, the process provides the compound of Formula (II-B) in a synthetic yield of greater than about 80%. In some embodiments, the process provides the compound of Formula (II-B) in a synthetic yield of greater than about 85%.

In some embodiments, the compound of Formula (II-F)

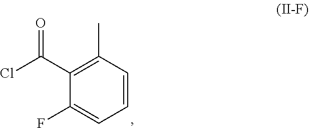

is synthesized by reacting a compound of Formula (II-G)

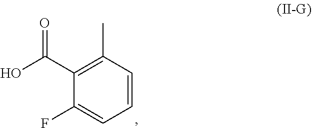

with an acyl halide preparation agent in the presence of a solvent.

In some embodiments, the acyl halide preparation agent is selected from oxalyl chloride, thionyl chloride, phosphoryl chloride, phosphorous trichloride, phosphorous pentachloride, phosgene, diphosgene, triphosgene, and cyanuric chloride. In some embodiments, the acyl halide preparation agent is oxalyl chloride. In some embodiments, the acyl halide preparation agent is thionyl chloride. In some embodiments, the acyl halide preparation agent is phosphoryl chloride. In some embodiments, the acyl halide preparation agent is phosphorous trichloride. In some embodiments, the acyl halide preparation agent is phosphorous pentachloride. In some embodiments, the acyl halide preparation agent is phosgene. In some embodiments, the acyl halide preparation agent is diphosgene. In some embodiments, the acyl halide preparation agent is triphosgene. In some embodiments, the acyl halide preparation agent is cyanuric chloride.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the compound of Formula (II-G), the acyl halide preparation agent, and the solvent are stirred:
for no longer than 16 hours; and
at a temperature of between about 20° C. and about 25° C.

### Examples

These examples are provided for illustrative purposes only and not to limit the scope of the claims provided herein. The starting materials and reagents used for the synthesis of the compounds described herein are synthesized or are obtained from commercial sources, such as, but not limited to, Sigma-Aldrich, Acros Organics, Fluka, and Fischer Scientific.

### Example 1: Synthesis of N-(5-Bromopyrazin-2-Yl)-2-Fluoro-6-Methylbenzamide (Compound 1.3)

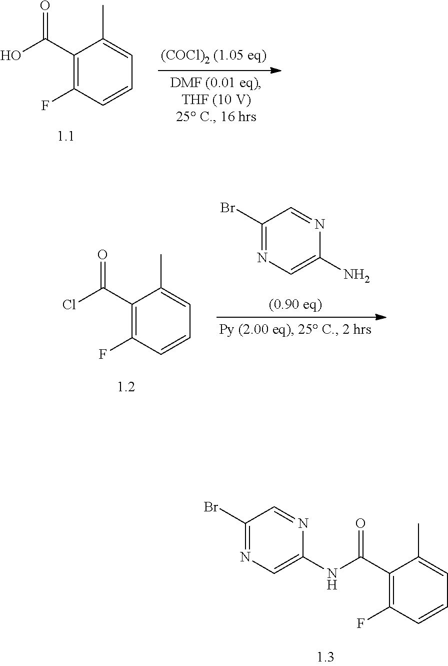 [SMILES: Cc1cccc(F)c1C(=O)O ; Cc1cccc(F)c1C(=O)Cl ; Nc1cnc(Br)cn1 ; Cc1cccc(F)c1C(=O)Nc1cnc(Br)cn1]

Step 1: Preparation of 2-fluoro-6-methylbenzoyl chloride (Compound 1.2)

In a 100 mL three-neck round bottom flask under N₂, Compound 1.1 (5.00 g, 32.4 mmol, 1.00 eq) and DMF (23.7 mg, 324.3 μmol, 0.01 eq) were dissolved in anhydrous THF (50 mL) at 25° C. The solution was cooled to 10-15° C. by cryogenic circulating bath and oxalyl chloride (4.32 g, 34.0 mmol, 1.05 eq) was added dropwise. The resulting reaction mixture was stirred at 20-25° C. for 16 hrs. Upon complete conversion of Compound 1.1, the reaction solution was carried forward to the next synthetic step without purification.

Step 2: Preparation of N-(5-bromopyrazin-2-yl)-2-fluoro-6-methylbenzamide (Compound 1.3)

5-bromopyrazin-2-amine (5.08 g, 29.2 mmol, 0.9 eq) was added to the THF solution of Compound 1.1 at 25° C. The reaction mixture was cooled to 0-5° C. by cryogenic circulating bath, and pyridine (5.13 g, 64.9 mmol, 2.00 eq) was added dropwise at 0-5° C. The resulting reaction mixture was stirred at 20-25° C. for 2 hrs. Upon complete conversion of Compound 1.2, ice water (50 mL) was added to the reaction solution with stirring at 20-25° C., and the mixture was extracted twice with EtOAc (20 mL, 10 mL). The combined organic phase was dried over Na₂SO₄, filtered, and concentrated under reduced pressure to give 15.4 g of crude Compound 1.3. The crude Compound 1.3 (15.4 g) was triturated with MTBE (60 mL) at 25° C. for 3 hrs, the suspension was filtered, and the filter cake was dried under reduced pressure to give Compound 1.3 (9.1 g, 26.4 mmol, 81% yield, 90% purity) as a gray solid.

### Example 2: Synthesis of 2-(6-chloro-2,2-difluorobenzo[d][1,3]dioxol-5-yl)-4,4,5,5-tetramethyl-1,3,2-dioxaborolane (Compound 2.3)

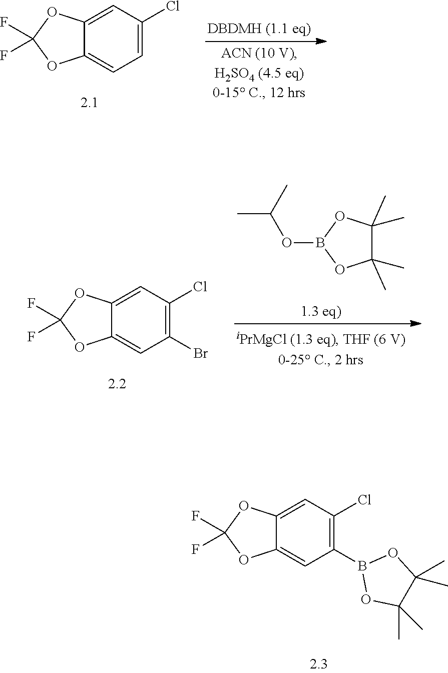 [SMILES: FC1(F)Oc2ccc(Cl)cc2O1 ; FC1(F)Oc2cc(Cl)c(Br)cc2O1 ; CC(C)OB1OC(C)(C)C(C)(C)O1 ; CC1(C)OB(c2cc3c(cc2Cl)OC(F)(F)O3)OC1(C)C]

Step 1: Preparation of 5-bromo-6-chloro-2,2-difluorobenzo[d][1,3]dioxole (Compound 2.2)

In a 100 mL three-neck round bottom flask, Compound 2.1 (3.00 g, 15.5 mmol, 1.00 eq) was dissolved in ACN (30 mL), and DBDMH (4.90 g, 17.1 mmol, 1.10 eq) was added at 25° C. The solution was cooled to 0-5° C. by cryogenic circulating bath and H₂SO₄ (6.8 g, 70.1 mmol, 4.50 eq) was added dropwise. The resulting reaction mixture was stirred at 10-15° C. for 12 hrs. Upon complete conversion of Compound 2.1, ice water (30 mL) was added dropwise to the reaction solution with stirring at 10-15° C. and the mixture was extracted with MTBE twice (10 mL, 5 mL). The combined organic phase was washed with 1M NaOH (20 mL). The organic phase was dried over Na₂SO₄, filtered, and concentrated under reduced pressure to give Compound 2.2 (3.60 g, 12.8 mmol, 82% yield, 97% purity) as a pale yellow liquid. Compound 2.2 was carried forward to the next synthetic step without purification.

Step 2: Preparation of 2-(6-chloro-2,2-difluorobenzo[d][1,3]dioxol-5-yl)-4,4,5,5-tetramethyl-1,3,2-dioxaborolane (Compound 2.3)

In a 100 mL three-neck round bottom flask under N₂, Compound 2.2 (3.60 g, 12.8 mmol, 1.00 eq) and 2-isopropoxy-4,4,5,5-tetramethyl-1,3,2-dioxaborolane (3.11 g, 16.7 mmol) was dissolved in anhydrous THF (21 mL) at 25° C. The solution was cooled to 0-5° C. by cryogenic circulating bath and isopropyl magnesium lithium chloride (1.30 M solution, 12.8 mL, 1.30 eq) was added dropwise. The resulting reaction mixture was stirred at 20-25° C. for 2 hrs. Upon complete conversion of Compound 2.2, ice water (50 mL) was added to the reaction solution with stirring at 20-25° C. The mixture was filtered through a pad of Celite, the filter cake was washed with MTBE twice (20 mL, 10 mL), and the filtrate was extracted twice with MTBE (10 mL, 5 mL). The combined organic phase was dried over Na₂SO₄, filtered, and concentrated under reduced pressure to give Compound 2.3 (3.87 g, 9.84 mmol, 76% yield, 81% purity) as a pale yellow waxy solid. Compound 2.3 was carried forward to the next synthetic step without purification.

### Example 3: Synthesis of N-(5-(6-chloro-2,2-difluorobenzo[d][1,3]dioxol-5-yl) pyrazin-2-yl)-2-fluoro-6-methylbenzamide (Compound 3.1)

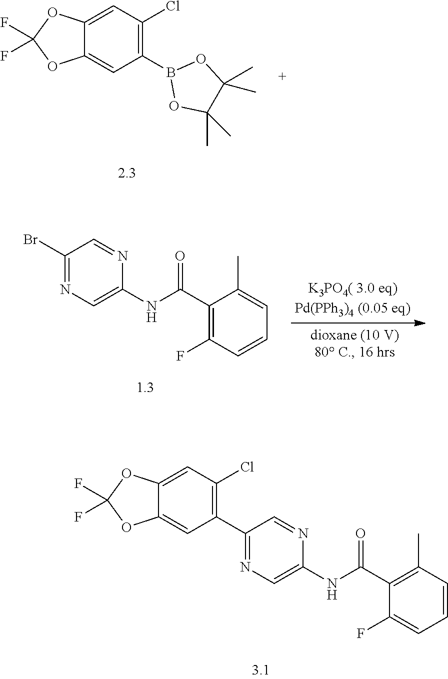 [SMILES: CC1(C)OB(c2cc3c(cc2Cl)OC(F)(F)O3)OC1(C)C ; Cc1cccc(F)c1C(=O)Nc1cnc(Br)cn1 ; Cc1cccc(F)c1C(=O)Nc1cnc(-c2cc3c(cc2Cl)OC(F)(F)O3)cn1]

In a 100 mL three-neck round bottom flask, Compound 2.3 (3.87 g, 9.84 mmol, 1.00 eq) and Compound 1.3 (3.56 g, 10.3 mmol, 1.05 eq) were dissolved in 1,4-dioxane (38 mL) at 25° C. under N₂. K₃PO₄ (6.27 g, 29.5 mmol, 3.00 eq) and Pd(PPh₃)₄ (568 mg, 492 μmol, 0.05 eq) were added at 25° C. in one portion. The resulting reaction mixture was de-gassed and stirred at 75-80° C. for 16 hrs. Upon complete conversion of Compound 2.3, the solution was concentrated and the resulting residue was added to MTBE (30 mL) and water (30 mL) and stirred for 30 min at 40° C. The mixture was extracted twice by MTBE (50 mL, 20 mL). The combined organic phase was dried over Na₂SO₄, filtered, and concentrated under reduced pressure to give 4.10 g of crude Compound 3.1. The crude Compound 3.1 was triturated with IPA/n-hexane (V/V=1:8, 50 mL) at 25° C. for 6 hrs, the resulting suspension was filtered, and the filter cake was dried under reduced pressure to give Compound 3.1 (3.3 g, 7.82 mmol, 79% yield, 100% purity) as an off-white solid.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-42. (canceled)

43. A process of synthesizing CRAC channel inhibitors of Formula (II): 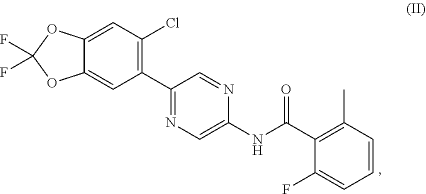 [SMILES: Cc1cccc(F)c1C(=O)Nc1cnc(-c2cc3c(cc2Cl)OC(F)(F)O3)cn1]
or a pharmaceutically acceptable salt thereof, wherein the process comprises reacting a compound of Formula (II-A) 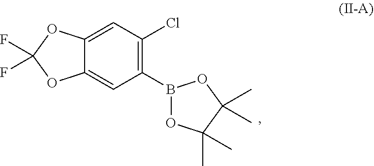 [SMILES: CC1(C)OB(c2cc3c(cc2Cl)OC(F)(F)O3)OC1(C)C]
with a compound of Formula (II-B) 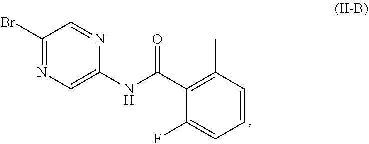 [SMILES: Cc1cccc(F)c1C(=O)Nc1cnc(Br)cn1]
in the presence of a base, a catalyst, and a solvent.

44. The process of claim 43, wherein the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine.

45. The process of claim 43, wherein the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄(BF₄)₂, PdCl₂, XPhos-Pd-G3, Pd-PEPPSI™—IPr, Pd-PEPPSI™—SIPr, and Pd-PEPPSI™-IPent.

46. The process of claim 43, wherein the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether.

47. The process of claim 43, wherein the compound of Formula (II-A), the compound of Formula (II-B), the base, the catalyst, and the solvent are stirred:
for no longer than 16 hours; and
at a temperature of between 75° C. and 80° C.

48. The process of claim 43, wherein the process provides the compound of Formula (II) in a synthetic yield of greater than 75%.

49. The process of claim 43, wherein the compound of Formula (II-A) 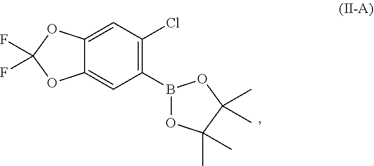 [SMILES: CC1(C)OB(c2cc3c(cc2Cl)OC(F)(F)O3)OC1(C)C]
is synthesized by reacting a compound of Formula (II-C) 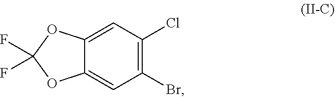 [SMILES: FC1(F)Oc2cc(Cl)c(Br)cc2O1]
with a borylating agent in the presence of a base and a solvent.

50. The process of claim 49, wherein the borylating agent is selected from 2-isopropoxy-4,4,5,5, -tetramethyl-1,3,2-dioxaborolane, 2-methoxy-4,4,5,5, -tetramethyl-1,3,2-dioxaborolane, and 2-ethoxy-4,4,5,5, -tetramethyl-1,3,2-dioxaborolane;
the base is selected from isopropylmagnesium chloride, isopropylmagnesium lithium chloride, methylmagnesium bromide, methylmagnesium chloride, methylmagnesium iodide, ethylmagnesium chloride, ethylmagnesium bromide, isopropylmagnesium bromide, methyllithium, ethyllithium, isopropyllithium, n-butyllithium, and tert-butyllithium; and
the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether.

51. The process of claim 49, wherein the compound of Formula (II-C), the borylating agent, the base, and the solvent are stirred:
for no longer than 2 hours; and
at a temperature of between 0° C. and 25° C.

52. The process of claim 49, wherein the process provides the compound of Formula (II-A) in a synthetic yield of greater than 70%.

53. The process of claim 49, wherein the compound of Formula (II-C) 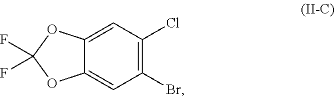 [SMILES: FC1(F)Oc2cc(Cl)c(Br)cc2O1]
is synthesized by reacting a compound of Formula (II-D) 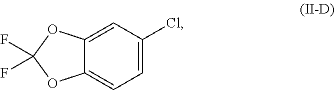 [SMILES: FC1(F)Oc2ccc(Cl)cc2O1]
with a brominating agent in the presence of an acid and a solvent.

54. The process of claim 53, wherein the brominating agent is selected from N-bromosuccinimide, tribromoisocyanuric acid, 1,3-dibromo-5,5-dimethylhydantoin, and bromine;
the acid is selected from hydrochloric acid, sulfuric acid, nitric acid, acetic acid, hydrobromic acid, phosphoric acid, formic acid, and trifluoroacetic acid; and
the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether.

55. The process of claim 53, wherein the compound of Formula (II-D), the brominating agent, the acid, and the solvent are stirred:
for no longer than 12 hours; and
at a temperature of between 0° C. and 15° C.

56. The process of claim 53, wherein the process provides the compound of Formula (II-C) in a synthetic yield of greater than 80%.

57. The process of claim 43, wherein the compound of Formula (II-B) 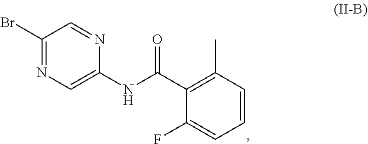 [SMILES: Cc1cccc(F)c1C(=O)Nc1cnc(Br)cn1]
is synthesized by reacting a compound of Formula (II-E) 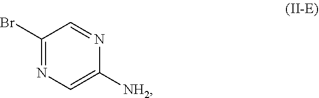
with a compound of Formula (II-F) 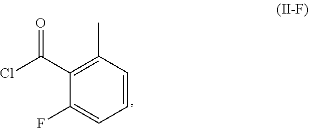
in the presence of a base.

58. The process of claim 57, wherein the base is selected from the group consisting of potassium carbonate, sodium carbonate, potassium bicarbonate, sodium bicarbonate, piperidine, pyridine, 1,8-diazabicyclo[5.4.0]undec-7-ene, sodium tert-butoxide, potassium tert-butoxide, cesium carbonate, potassium phosphate, sodium hydroxide, N,N-diisopropylethylamine, and triethylamine.

59. The process of claim 57, wherein the compound of Formula (II-E), the compound of Formula (II-F), and the base are stirred:
for no longer than 2 hours; and
at a temperature of between 20° C. and 25° C.

60. The process of claim 57, wherein the process provides the compound of Formula (II-B) in a synthetic yield of greater than 80%.

61. The process of claim 57, wherein the compound of Formula (II-F) 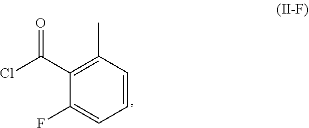
is synthesized by reacting a compound of Formula (II-G) 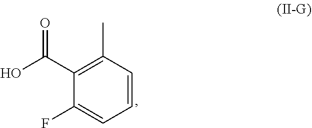
with an acyl halide preparation agent in the presence of a solvent.

62. The process of claim 61, wherein the acyl halide preparation agent is selected from oxalyl chloride, thionyl chloride, phosphoryl chloride, phosphorous trichloride, phosphorous pentachloride, phosgene, diphosgene, triphosgene, and cyanuric chloride; and
the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether.

63. The process of claim 61, wherein the compound of Formula (II-G), the acyl halide preparation agent, and the solvent are stirred:
for no longer than 16 hours; and
at a temperature of between 20° C. and 25° C.
